(19) United States
(12) Patent Application Publication
(10) Pub. No.: US 2026/0000608 A1
(43) Pub. Date: Jan. 1, 2026

(54) COMPOSITIONS AND METHODS FOR ADMINISTERING ANESTHETICS

(71) Applicant: Children's Medical Center Corporation, Boston, MA (US)

(72) Inventors: Daniel S. Kohane, Newton, MA (US); Tianjiao Ji, Boston, MA (US); Christopher B. Weldon, Brookline, MA (US)

(73) Assignee: Children's Medical Center Corporation, Boston, MA (US)

(21) Appl. No.: 19/259,065

(22) Filed: Jul. 3, 2025

Related U.S. Application Data
(62) Division of application No. 17/284,171, filed on Apr. 9, 2021.
(60) Provisional application No. 62/745,098, filed on Oct. 12, 2018.

(51) Int. Cl. A61K 9/1271; A61K 9/00; A61K 9/107; A61K 31/167; A61K 31/445

(52) U.S. Cl. CPC A61K 9/1271; A61K 9/0019; A61K 9/1075; A61K 31/167; A61K 31/445

(57) ABSTRACT

Compositions and methods of use related to formulations comprising anesthetics are generally described. Some embodiments are directed to compositions comprising a plurality of micelles and/or particles, and an anesthetic contained internally. These can be used to control and/or prolong the duration of IVRA while reducing the risk of systemic toxicity commonly due to administering anesthetics. The control and/or prolonged duration of IVRA may be due, at least in part, to the attachment of the sufficiently small micelles and/or particles to a biointerface (e.g., blood vessel surface) where the composition has been administered. Conventional IVRA methods commonly do not utilize potent and long-acting anesthetics (e.g., bupivacaine) due to the risks of cardiac toxicity. The compositions and methods described herein, however, provide a pathway for increased safety and efficiency of the use of such anesthetics, in certain embodiments. Resultantly, the performance (e.g., anesthetic distribution) of the micelles and/or particles internally containing an anesthetic may be comparatively better than the performance of free anesthetic, e.g., with respect to nerve blood and systematic drug distribution.

### RELATED APPLICATIONS

This application claims the benefit of U.S. Provisional Patent Application Ser. No. 62/745,098, filed Oct. 12, 2018, which is incorporated herein by reference in its entirety.

### GOVERNMENT FUNDING

This invention was made with government support under Grant. No. GM 073626 awarded by National Institutes of Health. The government has certain rights in the invention.

### TECHNICAL FIELD

Compositions and methods of use related to formulations comprising anesthetics are generally described.

### BACKGROUND

Intravenous regional anesthesia (IVRA) for surgery was first described in the early twentieth century, and is eponymously known as a Bier block. Anesthesia is administered by the intravenous injection of local anesthetics in a previously exsanguinated extremity, isolated from the rest of the circulation by a tourniquet. This procedure is a widely-accepted technique well suited for relatively brief surgeries on the extremities. IVRA has well-established drawbacks, however. The duration of IVRA is limited by concerns over limb ischemia due to continuous inflation of the cuff. Moreover, continuous inflation of the cuff is painful, and may necessitate deep sedation or terminating the Bier block and initiating general anesthesia. Deflation of the cuff, especially if inadvertent or premature, can release a large amount of local anesthetic into the systemic circulation, which can cause severe toxicity. Furthermore, IVRA does not necessarily provide postoperative pain relief, resulting in the use of systemic medications (e.g., opioids) or regional anesthetic techniques.

Accordingly, improved compositions and methods related to IVRA are needed.

### SUMMARY

Compositions and methods of use related to formulations comprising anesthetics are generally described.

In certain embodiments, a composition is described, wherein the composition comprises a plurality of micelles and/or particles comprising PEGylated lipids and/or polymers, and an anesthetic contained internally of the micelles and/or particles. In some embodiments, the micelles and/or particles have an average cross-sectional diameter of less than or equal to 30 nm.

Some embodiments are related to a composition comprising a plurality of micelles and/or particles having an average cross-sectional diameter of less than or equal to 30 nm, and an anesthetic contained internally within the plurality of micelles and/or particles.

According to certain embodiments, a method of delivering an anesthetic to a subject is described, wherein the method comprises decreasing blood circulation in an extremity of a subject by at least 50%, intravenously administering a composition comprising a plurality of micelles and/or particles to the extremity, the micelles and/or particles internally containing an anesthetic, wherein at least 50 mass % of the micelles and/or particles attach to a blood vessel surface within the extremity, and restoring blood circulation in the extremity of the subject.

In some embodiments, a method of delivering an anesthetic to a subject is described, wherein the method comprises applying a tourniquet to an extremity of the subject, administering a composition to the extremity, the composition comprising a plurality of micelles and/or particles having an average cross-sectional diameter of less than or equal to 30 nm, and internally containing an anesthetic, and removing the tourniquet from the extremity.

According to some embodiments, a method of delivering a drug to a subject is described, the method comprising applying a tourniquet to an extremity of the subject, administering a composition to the extremity, the composition comprising a plurality of micelles and/or particles having an average cross-sectional diameter of less than or equal to 30 nm, and internally containing a drug, and removing the tourniquet from the extremity.

In certain embodiments, a method of delivering a drug to a subject is described, wherein the method comprises decreasing blood circulation in an extremity of a subject by at least 50%, intravenously administering a composition comprising a plurality of micelles and/or particles to the extremity, the micelles and/or particles internally containing a drug, wherein at least 50 mass % of the micelles and/or particles attach to a blood vessel surface within the extremity, and restoring blood circulation in the extremity of the subject.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic of a method of delivering an anesthetic to a subject, and the interaction of micelles and/or particles with a blood vessel surface, according to certain embodiments;

FIG. 2A shows, according to certain embodiments, drug release kinetics of a non-limiting micellar composition comprising bupivacaine;

FIG. 2B shows, according to certain embodiments, transmission electron microscopy (TEM) images of a non-limiting micellar composition comprising bupivacaine;

FIG. 3 shows, according to certain embodiments, cytotoxicity of non-limiting micellar compositions comprising various bupivacaine concentrations;

FIG. 4A shows, according to certain embodiments, confocal laser scanning microscopy (CLSM) images of human umbilical vascular endothelial cells (HUVECs) incubated with a non-limiting micellar composition comprising bupivacaine;

FIG. 4B shows, according to certain embodiments, the fluorescent intensity of a non-limiting micellar composition comprising bupivacaine before and after incubation with HUVECs;

FIG. 5A shows, according to certain embodiments, the time course of fluorescence in a rat tail treated with a non-limiting micellar composition comprising bupivacaine;

FIG. 5B shows, according to certain embodiments, the quantification of the percentage of fluorescence intensity from FIG. 5A at each time point;

FIG. 6 shows, according to certain embodiments, representative images of the distribution of a non-limiting rhodamine (Rhd)-labeled micellar composition fifteen minutes after releasing the tourniquet;

FIG. 7A shows, according to certain embodiments, representative images of the distribution of a non-limiting Rhd-labeled micellar composition four hours after releasing the tourniquet;

FIG. 7B shows, according to certain embodiments, the ratio of total Rhd signal for a non-limiting micellar composition comprising bupivacaine at four hours (as shown in FIG. 7A) to 15 minutes (as shown in FIG. 6);

FIG. 8 shows, according to certain embodiments, a time course of tail analgesia duration for a non-limiting micellar composition comprising bupivacaine;

FIG. 9 shows, according to certain embodiments, the concentration of bupivacaine in blood after administering a non-limiting micellar composition comprising bupivacaine and releasing the tourniquet;

FIG. 10 shows, according to certain embodiments, the concentration of bupivacaine in blood after administering 0.5% free bupivacaine and releasing the tourniquet;

FIG. 11 shows, according to certain embodiments, the histological evaluation of H&E-stained sections 24 hour after treatment with a non-limiting micellar composition comprising bupivacaine; and

FIG. 12 shows, according to certain embodiments, H&E-stained sections of heart, liver, spleen, lung and kidney 24 hour after treatment with a non-limiting micellar composition comprising bupivacaine.

### DETAILED DESCRIPTION

Compositions and methods of use related to formulations comprising anesthetics are generally described. Some embodiments are directed to compositions comprising a plurality of micelles and/or particles, and an anesthetic contained internally. These can be used to control and/or prolong the duration of IVRA while reducing the risk of systemic toxicity commonly due to administering anesthetics. The control and/or prolonged duration of IVRA may be due, at least in part, to the attachment of the sufficiently small micelles and/or particles to a biointerface (e.g., blood vessel surface) where the composition has been administered. Conventional IVRA methods commonly do not utilize potent and long-acting anesthetics (e.g., bupivacaine) due to the risks of cardiac toxicity. The compositions and methods described herein, however, provide a pathway for increased safety and efficiency of the use of such anesthetics, in certain embodiments. Resultantly, the performance (e.g., anesthetic distribution) of the micelles and/or particles internally containing an anesthetic may be comparatively better than the performance of free anesthetic, e.g., with respect to nerve blood and systematic drug distribution.

Without wishing to be bound by theory, it is believed that conventional IVRA techniques utilizing a tourniquet may result in severe pain to the subject and/or limb ischemia. In certain cases, such IVRA techniques may require deep sedation of the subject in order to account for the severe pain. Additionally, conventional IVRA techniques utilizing local anesthetics may result in severe toxicity as a result of either prolonged anesthesia or unplanned release of the tourniquet. Some of the methods described herein can be used to administer a composition to an extremity of a subject such that the composition comprising micelles and/or particles and an anesthetic internally contained within the micelles and/or particles attach or otherwise bind to the blood vessel surface and release the anesthetic in a safe manner. More specifically, a significantly low concentration of the anesthetic may exist in the blood after the administration of the anesthetic. Thus, in certain embodiments, the compositions and methods can be used to provide IVRA to an extremity of a subject for longer durations as compared to conventional methods.

One set of embodiments is generally directed to compositions comprising a plurality of micelles and/or particles. The plurality of micelles and/or particles may comprise any of a variety of suitable species. For example, in certain embodiments, the plurality of micelles and/or particles may comprise silica based micelles and/or particles (e.g., silica based nanoparticles, silica and/or organosilica cross-linked micelles and/or polymers). In certain embodiments, the plurality of micelles and/or particles may comprise lipids and/or polymers. For instance, the plurality of micelles and/or particles may comprise dendritic lipids and/or polymers. In some cases, the plurality of micelles and/or particles may comprise lipids and/or polymers that are functionalized and/or conjugated with polyethylene glycol (PEG). Such lipids and/or polymers are abbreviated herein as PEGylated lipids and/or polymers. The PEGylated lipids and/or polymers may be at least partially hydrophobic.

The PEGylated lipids and/or polymers may comprise any of a variety of suitable species. For example, in some aspects, the PEGylated lipids and/or polymers comprise 2-distearoyl-sn-glycero-3-phosphoethanolamine conjugated PEG (DSPE-PEG), 1,2-dipalmitoryl-sn-glycero-3-phosphoethanolamine conjugated PEG (DPPE-PEG), N-(methylpolyoxyethylene oxycarbonyl)-1,2-dimyristoyl-sn-glycero-3-phosphoethanolamine conjugated PEG (DMPE-PEG), polyethylene glycol-poly lactic acid-co-glycolic acid (PEG-PLGA), and/or polyethylene glycol-polylactic acid (PEG-PLA). In certain embodiments, the PEGylated lipids and/or polymers may cause the composition to have a substantially statically neutral charge, a slightly statically negative charge (e.g., the PEGylated lipids and/or polymers comprise a slightly negative Zeta potential), or a slightly statically positive charge (e.g., the PEGylated lipids and/or polymers comprise a slightly positive Zeta potential).

The PEGylated lipids and/or polymers may have any of a variety of suitable molecular weights. In certain embodiments, the PEGylated lipids and/or polymers have a molecular weight in the range of 200 Daltons (Da) to 5,000 Da. In some cases, the PEGylated lipids and/or polymers may have a molecular weight of greater than or equal to 200 Da, greater than or equal to 500 Da, greater than or equal to 1,000 Da, greater than or equal to 1,500 Da, greater than or equal to 2,000 Da, greater than or equal to 2,500 Da, greater than or equal to 3,000 Da, greater than or equal to 3,500 Da, greater than or equal to 4,000 Da, or greater than or equal to 4,500 Da. In some embodiments, the PEGylated lipids and/or polymers may have a molecular weight of less than or equal to 5,000 Da, less than or equal to 4,500 Da, less than or equal to 4,000 Da, less than or equal to 3,500 Da, less than or equal to 3,000 Da, less than or equal to 2,500 Da, less than or equal to 2,000 Da, less than or equal to 1,500 Da, less than or equal to 1,000 Da, or less than or equal to 500 Da. Combinations of the above recited ranges are also possible (e.g., the PEGylated lipids and/or polymer have a molecular weight of greater than or equal to 500 Da and less than or equal to 4,000 Da, the PEGylated lipids and/or polymers have a molecular weight of greater than or equal to 1,500 Da and less than or equal to 2,500 Da). For example, in a certain, non-limiting embodiment, the PEGylated lipids and/or polymers comprise DSPE-PEG with a molecular weight of about 2000 Da.

In certain embodiments, the composition may comprise an anesthetic (e.g., a local anesthetic). The anesthetic may be contained internally of the micelles and/or particles. In a non-limiting example, the anesthetic may be internally contained within the core (e.g., hydrophobic core) of the micelles and/or particles comprising PEGylated lipids and/or polymers. In some cases, however, no PEGlyated lipids and/or polymers may be present. One or more than one anesthetic may be present within the composition. Also, in some cases, other drugs or pharmaceutically active agents may also be included within the composition (e.g., in addition or instead of an anesthetic).

According to some embodiments, the local anesthetic comprises an amide-containing local anesthetic (e.g., an amino-amide anesthetic). For example, in some embodiments, the local anesthetic may comprise articaine, bupivacaine, cinchocaine, etidocaine, levobupivacaine, lidocaine, mepivacaine, prilocaine, ropivacaine, and/or trimecaine. in one embodiment, the local anesthetic may be bupivacaine. In another embodiment, the local anesthetic may be lidocaine. The local anesthetic may be in a basic form (e.g., bupivacaine free base) or an acidic form (e.g., bupivacaine hydrochloride). In some non-limiting embodiments, it may be particularly useful to employ a local anesthetic in a basic form (e.g., bupivacaine free base) in order to internally contain the anesthetic in the hydrophobic core of the micelles and/or particles comprising PEGylated lipids and/or polymers. In addition, in certain embodiments, the local anesthetic comprises an amino-ester anesthetic. For example, the local anesthetic may comprise, benzocaine, chloroprocaine, cocaine, cyclomethycaine, dimethocaine, piperocaine, propoxycaine, procaine, and/or tetracaine. In some embodiments, more than one anesthetic may be present. For example, the composition may comprise bupivacaine and lidocaine, bupivacaine and another anesthetic, lidocaine and another anesthetic, etc.

According to some embodiments, the local anesthetic may comprise saxitoxin, neosaxitoxin, tetrodotoxin, menthol, eugenol, spilanthol, iontocaine, epinephrine, adrenaline, a vasoconstrictor, an adjuvant compound, a capsinoid, and/or a sodium channel blocker (e.g., a site 1 sodium channel blocker).

According to certain embodiments, the composition may be formulated via any of a variety of suitable methods for administration to a subject. For example, the composition may be formulated via a film dispersion method or a film hydration method. In certain embodiments, the composition may be formulated by nanoprecipitation. The local anesthetic (e.g., bupivacaine), may be loaded internally of the micelles and/or particles during the formulation of the composition (e.g., during film dispersion and/or during nanoprecipitation).

In a non-limiting embodiment, the composition can be formulated via a film dispersion method by dissolving PEGylated lipids and anesthetic in a solution, removing the solution to form a lipid-containing film, and hydrating the dried film.

The composition may comprise the local anesthetic (e.g., internally contained within the micelles and/or particles) in any of a variety of suitable amounts. Without wishing to be bound by theory, it is believed that a suitable amount of anesthetic (e.g., 10 wt. %) can be used in order to effectively provide anesthesia to a subject upon administering the composition. According to some embodiments, the composition comprises the local anesthetic in an amount of greater than or equal to 5 wt. %. For example, the composition may comprise the local anesthetic in an amount of greater than or equal to 10 wt. %, greater than or equal to 15 wt. %, greater than or equal to greater than or equal to 20 wt. %, or greater than or equal to 25 wt. %. In some embodiments, the composition may comprise the local anesthetic in an amount of less than or equal to 30 wt. %, less than or equal to 25 wt %, less than or equal to 20 wt. %, less than or equal to 15 wt. %, or less than or equal to 10 wt. %. Combinations of the above recited ranges are also possible (e.g., the composition comprises the local anesthetic in an amount of greater than or equal to 5 wt. % and less than or equal to 30 wt. %, composition comprises the local anesthetic in an amount of greater than or equal to 10 wt. % and less than or equal to 15 wt. %). In certain non-limiting embodiments, the composition may comprise the local anesthetic in an amount greater than 30 wt. % (e.g., 40 wt. %, 50 wt. %, 60 wt. %, 70 wt. %, 80 wt. %, etc.). The amount of local anesthetic contained internally of the micelles and/or particles may be determined via methods known to those of ordinary skill in the art, such as high-performance liquid chromatography (HPLC). As an example, in a certain, non-limiting embodiment, the composition comprises bupivacaine in an amount of about 10 wt. %.

Without wishing to be bound by any theory, it may be beneficial for the composition to comprise additional components depending on the application of the composition (e.g., the reason for administering anesthesia). In some embodiments, the additional components may comprise additional anesthetics. In certain aspects, the composition may comprise an additive, such as a salt (e.g., NaCl), an organic acid, a peptide (e.g., a cell penetrating peptide) a protein, a steroid, and/or a hyperpolarization-activated cation channel blocker, and the like.

According to certain embodiments, the micelles and/or particles may have any of a variety of suitable forms (e.g., structures, sizes, and/or shapes). For example, at least a portion of the micelles and/or particles may be in the form a nanostructure (e.g., a nanoparticle, nanowire, nanosheet, nanofilm, nanocone, nanopillar, nanorod, and the like). “Nanostructure” is used herein in a manner consistent with its ordinary meaning in the art. In certain embodiments, a nanostructure has a characteristic dimension, such as a cross-sectional diameter, or other appropriate dimension, that is between or equal to 1 nm and 1 micrometer.

According to certain embodiments, the micelles and/or particles (e.g., nanoparticles) may have any suitable average characteristic dimension (e.g., cross-sectional diameter). In some embodiments, the micelles and/or particles may have an average characteristic dimension (e.g., cross-sectional diameter) of less than or equal to 30 nm or less when the anesthetic is contained internally of the micelles and/or particles. For example, the micelles and/or particles may have an average characteristic dimension (e.g., cross-sectional diameter) of less than or equal to 25 nm, less than or equal to 20 nm, or less than or equal to 15 nm when the anesthetic is contained internally of the micelles and/or particles. According to certain aspects, the micelles and/or particles may have an average characteristic dimension (e.g., cross-sectional diameter) of greater than or equal to 10 nm, greater than or equal to 15 nm, greater than or equal to 20 nm, or greater than or equal to 25 nm when the anesthetic is contained internally of the micelles and/or particles. Combinations of the above recited ranges are also possible (e.g., the micelles and/or particles have an average characteristic dimension (e.g., cross-sectional diameter) of less than or equal to 30 nm and greater than greater than or equal to 10 nm when the anesthetic is contained internally of the micelles and/or particles, the micelles and/or particles have an average characteristic dimension (e.g., cross-sectional diameter) of less than or equal to 20 nm and greater than or equal to 15 nm when the anesthetic is contained internally of the micelles and/or particles).

There may be an increase in the average characteristic dimension (e.g., cross-sectional diameter) of the micelles and/or particles internally containing an anesthetic as compared to theoretical micelles and/or particles that do not contain an anesthetic in some cases. In certain embodiments, the average characteristic dimension (e.g., cross-sectional diameter) of the micelles and/or particles internally containing an anesthetic may increase by about 1 nm, about 2 nm, about 3 nm, about 4 nm, about 5 nm, about 6 nm, about 7 nm, about 8 nm, about 9 nm, or about 10 min as compared to theoretical micelles and/or particles that do not contain an anesthetic.

In some embodiments, the structure, size (e.g., average cross-sectional diameter), and/or shape of the micelles and/or particles can be measured by spectroscopic techniques, including dynamic light scattering (DLS), scanning electron microscopy (SEM), and/or TEM. The spectroscopic techniques can be supplemented by, for example, profilometry (e.g., optical or contact profilometers).

It may be beneficial for the micelles and/or particles to be in the form of a nanostructure (e.g., a nanoparticle) with a high surface area-to-volume ratio so that the micelles and/or particles have the ability to attach or otherwise bind to (for example, via specific or non-specific binding) a blood vessel surface upon administration to an extremity of a subject. Accordingly, in some embodiments, the composition has a surface area-to-volume ratio of greater than or equal to about 0.10 and less than or equal to about 0.70. For example the composition may have a surface area-to-volume ratio of greater than about 0.10. greater than about 0.20, greater than about 0.30, greater than about 0.40, greater than about 0.50, or greater than about 0.60. In certain embodiments, the composition has a surface area-to-volume ratio of less than or equal to 0.70, less than or equal to 0.60, less than or equal to 0.50, less than or equal to 0.40, less than or equal to 0.30, or less than or equal to 0.20. Combinations of the above recited ranges are also possible (e.g., the composition has a surface area-to-volume ratio of greater than about 0.20 and less than about 0.60, the composition has a surface area-to-volume ratio of greater than about 0.40 and less than about 0.50).

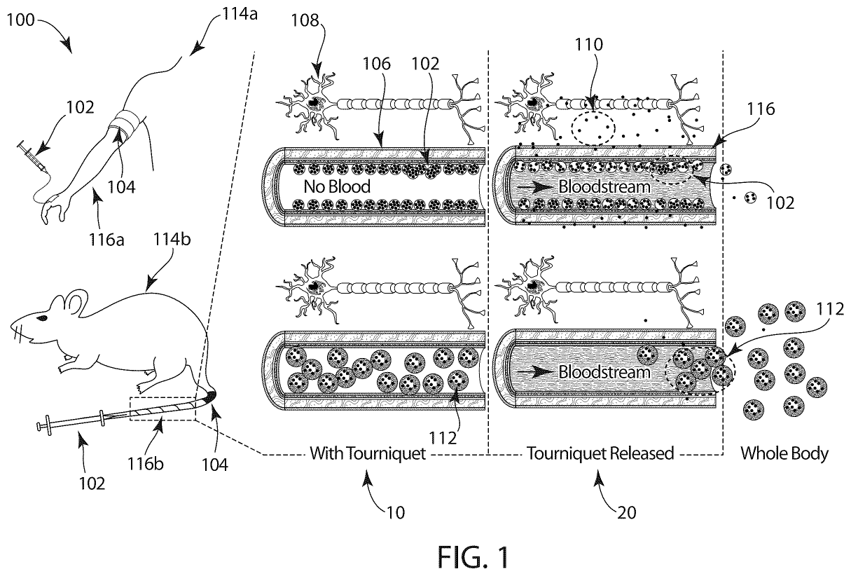

In certain embodiments, methods of delivering an anesthetic to a subject are described. FIG. 1 shows a schematic of a method of delivering an anesthetic to a subject, and the interaction of micelles and/or particles with a blood vessel surface, according to certain embodiments. Referring to FIG. 1, method 100 may be IVRA.

According to certain embodiments related to intravenous anesthesia, it is beneficial to decrease blood circulation where the intravenous anesthesia will take place so as to properly administer the composition without releasing the anesthetic into the blood stream. Accordingly, some embodiments may comprise decreasing blood circulation in an extremity of a subject. For example, decreasing blood circulation in the extremity of the subject may comprise applying a tourniquet to the extremity of the subject. As shown in FIG. 1, method 100 may comprise decreasing blood circulation in extremity 116 of subject 114 comprising applying tourniquet 104 to extremity 116 of subject 114. The tourniquet may be tightly wrapped around the extremity of the subject. In some aspects, an elastic tourniquet or a rubber tourniquet may be used to decrease blood circulation in the extremity. Other methods may also be used in some cases to decrease blood circulation, including administering suitable drugs. In some embodiments, the subject may be exposed to general anesthesia (e.g., isoflurane with an oxygen carrier gas) prior to decreasing the blood circulation in the extremity.

The blood circulation in the extremity of the subject may be decreased by any of a variety of suitable amounts. For example, in certain embodiments, the blood circulation in the extremity of the subject may be decreased by at least 95%, at least 90%, at least 85%, at least 80%, at least 75%, at least 70%, at least 65%, at least 60%, at least 55%, at least 50%, at least 45%, at least 40%, at least 35%, at least 30%, or at least 25% (i.e., as measured by volumetric flow). In some embodiments, the blood circulation in the extremity of the subject may be decreased by at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%. Combinations of the above recited ranges are also possible (e.g., the blood circulation in the extremity of the subject may be decreased by between at least 25% and at most 100%, the blood circulation in the extremity of the subject may be decreased by between at least 40% and at most 60%). According to certain embodiments, the percent decrease in the blood circulation in the extremity of the subject depends on how tight and/or how long the tourniquet is applied, which may vary depending on the subject.

The subject may be any of a variety of suitable subjects. The subject, for example, may be a human (e.g., 114a in FIG. 1). In other embodiments, the subject is an animal, such as a rat (e.g., 114b in FIG. 1), a mouse, and the like. Accordingly, the extremity of the subject may be any suitable extremity. For example, in the case of a human, the extremity may be an arm (e.g., 116a in FIG. 1) or a leg. In the case of an animal (e.g., a rat), the extremity may be an arm, leg, or tail (e.g., 116b in FIG. 1). It should be noted that the above mentioned subjects and extremities are only representative examples and other subjects and extremities are also possible, as would be understood by one of ordinary skill in the art.

According to certain embodiments, a method may comprise administering the composition comprising micelles and/or particles internally containing the anesthetic to the extremity, e.g., intravenously (IV) or intraarterially. The composition may be administered to the extremity via any of a variety of suitable methods that would be known to one of ordinary skill in the art. For example, in certain embodiments, the composition may be present in solution (e.g., aqueous solution) and administered to the extremity by injection (e.g., with an IV catheter). As shown in FIG. 1, composition 102 may be administered to subject 114 by injection.

In certain embodiments, after intravenously administering the composition, the micelles and/or particles may attach to a blood vessel surface within the extremity. The blood vessel surface may be a blood vein surface. The blood vessel surface may also (and/or alternatively) be an artery and/or a capillary. As shown in Part 10 of FIG. 1, for example, composition 102 comprising micelles and/or particles may attach to blood vessel surface (e.g., blood vein surface) 106. As is explained below in greater detail, the attachment may occur, for example, due to the decreased blood flow in the extremity, the size of the micelles and/or particles, and/or hydrogen-bonding between the micelles and/or particles and the blood vessel surface.

Any of a variety of suitable amount of the micelles and/or particles may attach to the blood vessel surface within the extremity. In certain embodiments, at least 75 mass %, at least 70 mass %, at least 65 mass %, at least 60 mass %, at least 55 mass %, at least 50 mass %, at least 45 mass %, at least 40 mass %, at least 35 mass %, at least 30 mass %, or at least 25 mass % of the micelles and/or particles attach to the blood vessel surface within the extremity. In some embodiments, at most 25 mass %, at most 30 mass %, at most 35 mass %, at most 40 mass %, at most 45 mass %, at most 50 mass %, at most 55 mass %, at most 60 mass %, at most 65 mass %, at most 70 mass %, or at most 75 mass % of the micelles and/or particles attach to the blood vessel surface within the extremity. Combinations of the above recited ranges are also possible (e.g., between at least 25% and at most 75% of the micelles and/or particles attach to a blood vessel surface within the extremity, between at least 40% and at most 60% of the micelles and/or particles attach to a blood vessel surface within the extremity).

According to certain embodiments, the amount of micelles and/or particles that attach to the blood vessel surface within the extremity may be determined using fluorescent labeling and an imaging system, such as an In-Vivo Imaging System (IVIS). For example, in certain embodiments, the micelles and/or particles may be fluorescently labeled with a dye, such as Rhd. Upon administering the composition, fluorescent spectroscopy may be used in some embodiments to determine the amount of micelles and/or particles that attached to the blood vessel surface. Additionally, in certain aspects, the amount of micelles and/or particles that attach to the blood vessel surface within the extremity may be determined by microscopy techniques. However, it should be understood that in other embodiments, no labeling or imaging would be required.

The interaction between the micelles and/or particles and the blood vessel surface may occur to any of a variety of suitable factors and/or mechanisms, which may lead to attachment or binding. For example, in some embodiments, the interaction (e.g., attachment) between the micelles and/or particles and the blood vessel may occur, at least in part, due to the fact that the micelles and/or particles are administered to an extremity wherein the blood circulation in the extremity has been decreased (e.g., by 50%), as explained herein. For example, as shown in Part 10 of FIG. 1, composition 102 attaches to blood vessel surface 106 due at least in part to decreased blood circulation in extremity 116 of subject 114. In some embodiments, for example, the decrease in blood circulation in the extremity of the subject may cause the micelles and/or particles to statically isolate proximate to the blood vessel surface due the absence of the flow of blood.

In some conventional cases, formulations comprising an anesthetic for local anesthesia tend to be quite large (e.g., micron scale). The rationale has been that larger particles, having a smaller surface area-to-volume ratio, will have higher drug loading, and release drugs more slowly. For example, as shown in Part 10 of FIG. 1, theoretical formulation 112 may have a characteristic dimension (e.g., average cross-sectional diameter) that is comparatively larger than the average characteristic dimension of composition 102 (e.g., 100 nm vs. less than or equal to 30 nm, respectively). Resultantly, formulation 112 may be too large to attach to blood vessel surface 106. In certain embodiments, the micelles and/or particles may attach to the blood vessel surface due at least in part to the characteristic dimension (e.g., the average cross-sectional diameter of less than or equal to 30 nm) of the micelles and/or particles. For example, in some embodiments, the micelles and/or particles may attach to the blood vessel surface due at least in part to the substantially large surface area-to-volume ratio of the micelles and/or particles. In some embodiments, the substantially larger surface area-to-volume ratio may cause the micelles and/or particles to disperse substantially evenly along the blood vessel surface. Resultantly, and surprisingly, the substantially smaller compositions comprising micelles and/or particles and an anesthetic internally contained within the micelles and/or particles perform better, for example with respect to anesthetic distribution and/or duration of anesthesia, than substantially larger theoretical formulations (e.g., 112) and/or free anesthetic. In contrast, the expectation would have been that larger particles are more efficient at delivering anesthesia, due to their larger internal volumes.

In certain embodiments, the micelles and/or particles may attach or bind to the blood vessel surface due at least in part to various factors, such as electrostatic interactions, hydrogen-bonding, nonspecific binding, etc. between the micelles and/or particles and the blood vessel surface. For example, referring to Part 10 of FIG. 1, composition 102 may hydrogen-bond to blood vessel surface 106. Such hydrogen-bonding may occur, for example, between a hydrogen (H) atom and at least partially more electronegative atom, such as nitrogen (N), oxygen (O), or fluorine (F). Other types of bonding, such as electrostatic interactions, may also facilitate attachment and/or binding.

In some embodiments, upon attaching to the blood vessel surface, the micelles and/particles release the anesthetic (e.g., into the blood and/or into the blood vessel). In certain embodiments, the attachment of the micelles and/or particles to the surface may provide a local and/or sustained release of the anesthetic. Consequently, the release of the anesthetic may cause regional anesthesia in the extremity of the subject to occur. For example, as shown in Part 20 of FIG. 1, composition 102 releases anesthetic 110 into the blood vessel such that the anesthetic is proximate nerve 108 and causes regional anesthesia to nerve 108 in extremity 116 of subject 114. In certain embodiments, the release of the anesthetic occurs over the course at least 1 minute, at least two minutes, at least five minutes, at least ten minutes, or at least fifteen minutes after the composition is administered to the extremity of the subject.

The micelles and/or particles may release any suitable amount of the anesthetic into the extremity (e.g., upon attaching to the blood vessel surface), depending on the quantity of micelles and/or particles delivered, or attached to the blood vessel surface, etc.. For example, in some embodiments, the micelles and/or particles release greater than or equal to 50%, greater than or equal to 60%, greater than or equal to 70%, greater than or equal to 80%, greater than or equal to 90%, or 100% of the anesthetic into the extremity. In certain aspects, the micelles and/or particles release less than or equal to 100%, less than or equal to 80%, less than or equal to 70%, or less than or equal to 60% of the anesthetic into the extremity. Combinations of the above recited ranges are also possible (e.g., the micelles and/or particles release greater than or equal to 50% and less than or equal to 90% of the anesthetic into the extremity, the micelles and/or particles release greater than or equal to 90% and less than or equal to 100% of the anesthetic into the extremity. According to certain embodiments, the composition releases 90% of the anesthetic into the extremity. According to certain embodiments, the composition may release the anesthetic into the extremity over a certain period of time. This may start to occur before blood flow has been restored (e.g., by removing a tourniquet). The period of time may be dependent on how long the tourniquet is applied to the extremity of the subject. In some embodiments, for example, the composition may release the anesthetic into the extremity over the period of 1 minute, 5 minutes, 15 minutes, 30 minutes, 1 hour, 5 hours, 10 hours, 15 hours, 24, hours, or 48 hours.

In some embodiments, the method may further comprise restoring blood circulation in the extremity of the subject. In certain embodiments, restoring blood circulation in the extremity of the subject may comprise removing the tourniquet from the extremity of the subject. In some embodiments, for example, blood circulation in the extremity of the subject may be substantially completely restored (e.g., to at least 90%, at least 95%, at least 98%, at least 99%, 100%, etc.) after removing the tourniquet from the subject. As shown in Part 20 of FIG. 1, for example, blood circulation in extremity 116 may be restored by removing the tourniquet from extremity 116. The removal of the tourniquet may take place after the tourniquet has been applied for any of a variety of suitable durations. For example, in certain embodiments, removing the tourniquet from the extremity takes place fifteen minutes after applying the tourniquet to the extremity. Removing the tourniquet from the extremity may take place thirty minutes, one hour, two hours, three hours, four hours, or five hours after applying the tourniquet to the extremity. The restoration of blood may occur slowly or quickly, depending on the embodiment. For example, a tourniquet may be simply removed, or the tourniquet may be gradually loosened over a period of time, e.g., to prevent sudden surges of blood flow. The composition and methods may be particularly useful for providing the subject with a suitable and/or prolonged duration of anesthesia. For example, in some embodiments, the micelles and/or particles internally containing an anesthetic advantageously provide a controlled duration of anesthesia (e.g., upon attaching to the blood vessel surface and releasing the anesthetic). Such a prolonged and/or controlled duration of anesthesia can provide a safe and efficient way of administering an anesthetic to a subject, as compared to the administration of a free anesthetic and/or other theoretical formulations comprising an anesthetic.

According to certain embodiments, the IVRA lasts for at least 1 hour, at least 2 hours, at least 3 hours, at least 4 hours, at least 5 hours, at least 10 hours, or at least 24 hours upon administering the composition to the extremity of the subject. In some embodiments, the IVRA lasts for at most 48 hours, at most 24 hours, at most 10 hours, at most 5 hours, at most 4 hours, at most 3 hours, or at most 2 hours upon administering the composition to the extremity of the subject. Combinations of the above recited ranges are also possible (e.g., the IVRA lasts for at least 1 hour and at most 24 hours upon administering the composition to the extremity of the subject, the IVRA lasts for at least 3 hours and at most 5 hours upon administering the composition to the extremity of the subject).

A low concentration of the anesthetic in the blood circulation (e.g., the whole blood circulation) after restoring blood circulation in the extremity of the subject can be an important component regarding the safety of the anesthetic procedure in certain instances. In some cases, for example, if a high concentration (e.g., greater than or equal to 5 micrograms/mL) of anesthetic is left in the blood circulation after restoring blood circulation to the extremity of the subject, then anesthesia may dangerously persist in the extremity or an area proximate to the extremity (e.g., the anesthetic may enter systematic circulation throughout the rest of the subject's body). In addition, in some cases, a high concentration (e.g., greater than or equal to 5 micrograms/mL) of anesthetic may result in cytotoxicity and/or toxicity of the subject. It should be noted that 5 micrograms/mL is discussed here by way of example only, and that this is applicable to other concentrations as well.

According to certain embodiments, the concentration of the anesthetic in the blood circulation may be kept relatively low after restoring blood circulation in the extremity of the subject. For example, upon restoring blood circulation in the extremity of the subject, the concentration of anesthetic in the blood circulation is less than or equal to 5 micrograms/mL, less than or equal to 4 micrograms/mL, less than or equal to 3 micrograms/mL, less than or equal to 2 micrograms/mL, or less than or equal to 1 microgram/mL. In certain embodiments, upon restoring blood circulation in the extremity of the subject, the concentration of anesthetic in the blood circulation of the extremity is greater than or equal to 0 micrograms/mL, greater than or equal to 1 microgram/mL, greater than or equal to 2 micrograms/mL, greater than or equal to 3 micrograms/mL, or greater than or equal to 4 micrograms/mL. Combinations of the above recited ranges are also possible (e.g., the concentration of anesthesia in the blood circulation is less than or equal to 5 micrograms/mL and greater than or equal to 0 micrograms/mL upon restoring blood circulation in the extremity of the subject, the concentration of anesthesia in the blood circulation is less than or equal to 3 micrograms/mL and greater than or equal to 1 micrograms/mL).

The compositions and methods described herein may be particularly useful for any of a variety of medical procedures, including surgery. For example, in some embodiments, the compositions and methods may be applied in order to provide the subject with an effective amount of anesthesia in order to surgically operate on an extremity. Additionally, in certain embodiments, the compositions and methods described here may be used to investigate and/or treat certain disease states, including localized cancers and infections. For example, a method may comprise delivering a drug to a subject, wherein the drug comprises an anti-cancer drug, and treating the subject with the composition, wherein the subject has cancer and/or requires chemotherapy. In such instances, the ability to achieve very high local drug concentrations and retentions, while minimizing systemic drug levels is beneficial.

In general, the “effective amount” of a compound refers to an amount sufficient to elicit the desired biological response, e.g., an anesthetic response. As will be appreciated by those of ordinary skill in this art, the effective amount of a composition as discussed herein may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, the term “pharmaceutically active agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Pharmaceutically active agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. Preferably, though not necessarily, the pharmaceutically active agent is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference.

In some cases, the compositions may be applied to a subject, such a human subject. For instance, in one set of embodiments, the composition may be contained within a suitable needle or a syringe for injection into a subject, as discussed herein. Thus, another aspect provides a method of administering any composition discussed herein to a subject. When administered, the compositions may be applied in a therapeutically effective, pharmaceutically acceptable amount as a pharmaceutically acceptable formulation.

As used herein, the term “pharmaceutically acceptable” is given its ordinary meaning. Pharmaceutically acceptable compositions are generally compatible with other materials of the formulation and are not generally deleterious to the subject. Any of the compositions described herein may be administered to the subject in a therapeutically effective dose. The terms “treat,” “treated,” “treating,” and the like, generally refer to administration of the compositions described herein to a subject. When administered to a subject, effective amounts will depend on the particular condition being treated and the desired outcome. A therapeutically effective dose may be determined by those of ordinary skill in the art, for instance, employing factors such as those further described below and using no more than routine experimentation.

In administering the compositions to a subject, dosing amounts, dosing schedules, routes of administration, and the like may be selected so as to affect known activities of these compositions. Dosages may be estimated based on the results of experimental models, optionally in combination with the results of assays of the compositions discussed herein. Dosage may be adjusted appropriately to achieve desired drug levels, local or systemic, depending upon the mode of administration. The doses may be given in one or several administrations.

The dose of the composition to the subject may be such that a therapeutically effective amount of the composition reaches the active site (e.g., a blood vessel) within the subject, e.g., to cause anesthesia. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail with other pharmaceutically active agents), etc.

The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. In some cases a maximum dose can be used, that is, the highest safe dose according to sound medical judgment. In some cases, the dosage form is such that it does not substantially deleteriously affect the subject.

Administration of a composition as described herein may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of a composition within the subject without causing clinically unacceptable adverse effects.

Any medically acceptable method may be used to administer a composition to the subject. The administration may be localized (i.e., to a particular region, physiological system, tissue, organ, or cell type). The composition may be administered via injection in some cases. The composition also may be administered by other methods, e.g., through parenteral injection or implantation, via surgical administration, or any other method of administration where access to the target by a composition is achieved. Examples of parenteral modalities that can be used include intravenous, intradermal, subcutaneous, intracavity, intramuscular, intraperitoneal, epidural, or intrathecal. Examples of implantation modalities include any implantable or injectable drug delivery system.

In certain embodiments of the invention, the administration of a composition as discussed herein may be designed so as to result in sequential exposures to the composition over a certain time period, for example, minutes or hours. This may be accomplished, for example, by repeated administrations of a composition as described herein.

Administration of the composition can be alone, or in combination with other pharmaceutically active agents and/or compositions. In some embodiments, the compositions may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used.

Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable concentrations can be determined by those of ordinary skill in the art, using no more than routine experimentation. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of ordinary skill in the art can readily determine the various parameters for preparing and formulating various compositions as described herein without resort to undue experimentation.

Certain embodiments of the present invention also provides any of the above-mentioned compositions in kits, optionally including instructions for use of the composition. Instructions also may be provided for administering the composition by any suitable technique as previously described, for example, via injection or another known route of drug delivery. The kits described herein may also contain one or more containers, which may contain the inventive composition and other ingredients as previously described. The kits also may contain instructions for mixing, diluting, and/or administrating the compositions in some cases. The kits also can include other containers with one or more solvents, surfactants, preservative and/or diluents (e.g., normal saline (0.9% NaCl), or 5% dextrose) as well as containers for mixing, diluting or administering the compositions in a sample or to a subject in need of such treatment. U.S. Provisional Patent Application Ser. No. 62/745,098, filed Oct. 12, 2018 is incorporated herein by reference in its entirety. The following examples are intended to illustrate certain embodiments of the present invention, but do not exemplify the full scope of the invention.

### EXAMPLE 1

The following example describes the materials and methods for the preparation, characterization, and use of non-limiting compositions comprising a plurality of micelles and an anesthetic internally contained within the plurality of micelles.

### Preparation and Characterization

A non-limiting micellar composition comprising bupivacaine was prepared with 1,2-distearoyl-sn-glycero-3-phosphoethanolamine)-N-[(polyethylene glycol)-2000] (DSPE-PEG(2000), Avanti Polar Lipids, USA) and bupivacaine (Alfa Aesar, USA) via a film dispersion method. Bupivacaine (2 mg) and DSPE-PEG (2000) (10 mg) were dissolved in 10 mL of 9:1 chloroform:methanol (v/v) solution. The solvent was removed by vacuum rotary evaporation to form a dry bupivacaine-containing lipid film. The dried film was hydrated with saline at 60° C. for 30 minutes. Non-encapsulated bupivacaine was separated by centrifugation at 5,000 rpm. Size and zeta potential were determined by dynamic light scattering (DLS) (Delsa Nano; Beckman Coulter, USA). Drug loading was determined by high-performance liquid chromatography (HPLC; Agilent Technologies, USA) after dissolving the lyophilized micellar composition powder in acetonitrile (Sigma, USA). A fluorescent Rhd-labeled micellar composition was synthesized by mixing 0.5% (by weight) of the rhodamine B labeled lipid 1,2-dioleoyl-sn-glycero-3-phosphoethanolamine-N-(lissamine rhodamine B sulfonyl) (ammonium salt) (Avanti Polar Lipids, USA) DSPE-PEG (2000) before making the film. Bupivacaine was also added to the Rhd-labeled micellar composition, as described above, and the size of the Rhd-labeled micellar composition comprising bupivacaine was measured to be the same as that of the non-fluorescent counterpart. Micellar compositions without drug or dye were prepared in the same way, but with no drug/dye added. An alternate composition comprising liposomes and bupivacaine was synthesized for comparison to the micellar composition comprising bupivacaine. The liposomal composition was prepared by a film hydration method. In brief, a lipid mixture of 1,2-distearoyl-sn-glycero-3-phosphoethanolamine (DSPE) (Avanti Polar Lipids, USA), DSPE-PEG (2000), and cholesterol (Sigma, USA) in a 9:1:5 molar ratio was dissolved in a 9:1 chloroform/methanol (v/v) solution. The solvent of the lipid mixture was slowly evaporated under vacuum to form a lipid film. The lipid thin film was then dissolved in t-butanol and freeze-dried to form a lipid cake. A liposomal cake with no anesthetic was hydrated with saline at 60° C., and then underwent extrusion with a 200 nm membrane at room temperature. The liposomal composition comprising bupivacaine was formulated by hydrating the lipid cake with 250 mM (NH₄)₂SO₄ and then incubating with a 10 mg/mL bupivacaine hydrochloride (Sigma, USA) solution at 60° C. for 1 hour. After extrusion with a 200 nm membrane, the liposomes were dialyzed against saline for 48 hours at 4° C. The size of the liposomal compositions was determined by DLS. Drug loading was determined by HPLC after disrupting the liposomes with 100 mM octyl beta-D-glucopyranoside (Sigma, USA). For the preparation of a fluorescent Rhd-labeled liposomal composition, 0.5% (in whole weight) 1,2-dioleoyl-sn-glycero-3-phosphoethanolamine-N-(lissamine rhodamine B sulfonyl) (ammonium salt) was added to the lipid mixture before making the film.

### In Vitro Drug Release Kinetics

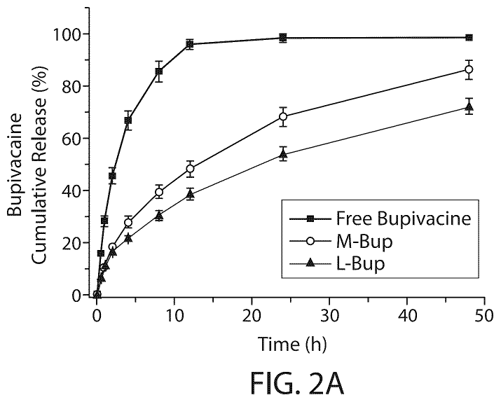

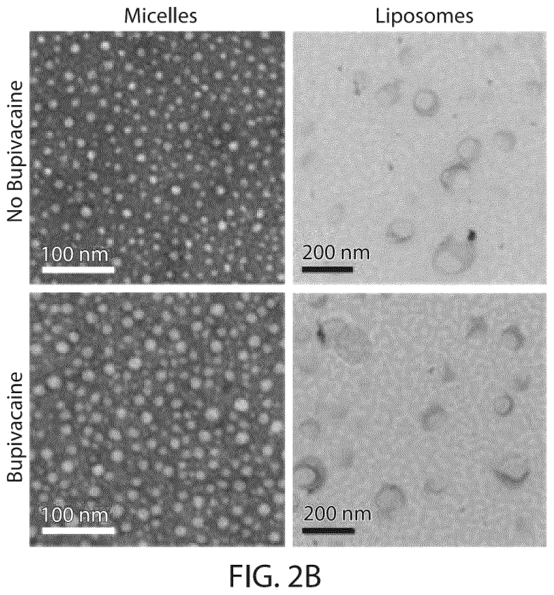

Drug release kinetics were performed by dialyzing 500 microliter solutions of: (i) free bupivacaine solution (1 mg/mL, w/v); (ii) micellar composition comprising bupivacaine (10 mg/mL, w/v); and (iii) liposomal composition comprising bupivacaine (10 mg/mL, w/v) against 14 mL of saline at 37° C. in a Slide-Alyzer MINI dialysis device (Thermo Fisher, USA) with a 20-kDa molecular weight cut-off. Samples were collected at predetermined time intervals and replaced with fresh saline. The concentration of drug in each sample was determined by HPLC (FIG. 2A).

### Cytotoxicity and Cell Interaction Behavior

HUVECs were purchased from ATCC. The cell line was authenticated by the suppliers and passaged in the laboratory for fewer than 3 months after resuscitation. HUVECs were maintained in a 37° C./5% CO₂ humidified chamber in Endothelial Cell Growth Medium (EBM-2) (Lonza, USA). When the cells grew to 80% confluence, the cell studies were performed. For cytotoxicity studies, HUVECs were seeded in 96-well plates and incubated with various drug formulations (free bupivacaine, micellar composition comprising bupivacaine, and liposomal composition comprising bupivacaine) for 24 hours. Cell viability was evaluated by CellTiter 96® AQueous One Solution Cell Proliferation Assay (MTS) (Promega, USA) according to suppliers' instructions. The absorbance at 490 nm wavelength was detected with a microplate reader (SYNERGYMx, BioTek®, USA). Untreated cells served as a control. Results were shown as the average cell viability [(OD treat OD blank)/(OD control OD blank)×100%] of quintuplicate wells. To investigate the interaction between formulations and cells, 3 mL of a 0.1 mg/mL Rhd-labeled micellar composition comprising bupivacaine or Rhd-labeled liposomal composition comprising bupivacaine (both of which had the same fluorescent intensity) were incubated with HUVECs in 6 cm diameter cell culture dishes for 15 minutes at 4° C., then the incubation medium was collected. The cells were washed three times with 3 mL of fresh medium and the washing medium was added to the collected incubated medium, the final volume of the collected medium was adjusted to 12 mL and the fluorescence intensity of each medium was measured by fluorescence spectrophotometer (Cary Eclipse, Agilent, USA). The excitation wavelength was 550 nm, and the collection range was 570-700 nm.

The washed cells were fixed with 4% paraformaldehyde (PFA), and the nuclei were stained with Hoechst 33342 (Thermo Fisher Scientific, USA) for 5 minutes. Cells were imaged under CLSM (Zeiss710, Germany), and the intensity of the Rhd fluorescence in each group was calculated from images using ImageJ.

### Animal Studies

Animal studies were performed with a protocol approved by the Boston Children's Hospital Animal Care and Use Committee that conform to the requirements of the International Association for the Study of Pain. Male Sprague-Dawley rats (Charles River Laboratories, USA) weighing 350-400 g were used for this study. Starting at 6 a.m., the rats were in light for 12 hours of each day.

In a typical IVRA procedures, the rats were anesthetized with isoflurane-oxygen. The tail vein was cannulated with a 24-gauge IV catheter, which was placed in the distal third of the tail and filled with heparinized saline (BD, USA). The tail was exsanguinated by wrapping it with a rubber strip. An elastic tourniquet was tightly applied proximal to the rubber strip and then the rubber strip was removed. Next, 0.4 mL of formulation was injected by a microinjector (1 mL) through the IV catheter. After releasing the tourniquet, animals were allowed to recover from the isoflurane anesthesia, then rat tail IVRA was assessed by the tail-flick test. In brief, tail-flick latency (the time from onset of radiant heat to tail-flick response) was measured by a tail-flick analgesia meter (Tail-Flick Unit 37370; Ugo Basile, Comerio, Italy). The intensity of radiant heat (parameter of the analgesia meter) was set at 90%, and cut-off time of radiant heat was set at 18 seconds. The testing area was between the injection site in the distal tail and the tourniquet, in the proximal tail. Baseline tail-flick latency was measured in untreated and saline injected rats. All evaluations were performed by two trained observers blinded to group allocation.

For in vivo imaging, the same IVRA procedures were performed, and 0.4 mL of: (i) free Rhd; Rhd-labeled micellar composition comprising bupivacaine; and Rhd-labeled liposomal composition comprising bupivacaine was injected into the tails of rats (n=4) after fastening the tourniquet. Images were taken with an In Vivo Imaging System (PerkinElmer Inc., USA) before releasing the tourniquet, and the signal intensity in those images was considered to be 100%. After releasing the tourniquet, images were taken at time points of 15 minutes, 1 hour, 4 hours, and 24 hours. The Rhd signals of each composition were quantified at each time point, and normalized to the intensity before tourniquet release.

For studies of distribution within the tail, rats were euthanized 15 minutes or 4 hours after releasing the tourniquet, and the tails were harvested and processed into frozen slices. Blood vessels were stained with a Rabbit monoclonal anti-CD34 antibody (ab81289, Abcam, USA) followed with Goat Anti-Rabbit IgG H&L (Alexa Fluor® 488) (ab150077, Abcam, USA). Nuclei were stained with Hoechst 33342. The slices were imaged by CLSM. The intensity of the Rhd fluorescence was calculated from images using ImageJ.

### Bupivacaine Concentration in the Blood

The following were administered as IVRA: saline, 0.5% free bupivacaine, 0.1% free bupivacaine, micellar composition comprising bupivacaine (0.1% bupivacaine), and liposomal composition comprising bupivacaine (0.1% bupivacaine). After releasing the tourniquet, 200 microliters of blood was collected (through the tail vein opposite from that through formulations were injected) at 15 minutes, 30 minutes, 45 minutes, 90 minutes, 150 minutes, 240 minutes, 480 minutes and 720 minutes. Blood was put in ethylenediaminetetraacetic acid (EDTA) containing blood collection tubes (BD, USA). The samples were stored in ice water for 10-15 minutes and then centrifuged at 3,000 rpm at 4° C. for 5 minutes to obtain plasma. The hemocytes were lysed by ultrasonication (Sonics & Materials, Inc., USA) at 4° C. for 2 minutes and centrifuged at 13,000 rpm at 4° C. for 5 minutes to obtain a supernatant. The supernatant was combined with plasma. Next, 50 microliters of the blood samples were mixed with 10 microliters of sodium hydroxide aqueous solution (1 M) and 0.3 mL ethyl ether in an ice-water bath. The mixtures were vigorously vortexed for 2 minutes and centrifuged at 3,000 rpm for minutes at room temperature. The organic phase was transferred to a 4 mL glass vial and evaporated to dryness in a 4° C. ice-water bath under vacuum. The residue was dissolved in 200 microliters of water-methanol (50/50, v/v), and was mechanically agitated for 30 seconds. The samples were transferred to clean polypropylene tubes and centrifuged at 13,000 rpm for 10 minutes. The supernatant was analyzed by HPLC.

### Histology

24 hours after the IVRA procedures, rats that had been administered saline, free bupivacaine (0.5% bupivacaine), micellar composition comprising bupivacaine (0.1% bupivacaine), and liposomal composition comprising bupivacaine (0.1% bupivacaine) were euthanized. Tails and organs (e.g., heart, liver, spleen, lung, and kidney) were harvested and fixed with 10% formalin. The organs were processed by standard procedures to produce hematoxylin and eosin-stained (H&E stained) slides. The tails were decalcified to remove bones before H&B staining procedures. Similar regions were examined in each organ from different groups. Tissues from healthy rats (no treatment) were used as negative controls.

### Statistical Analysis

Statistical analysis was conducted by the Student's t-test for comparison of 2 groups and one-way ANOVA for multiple groups, followed by Newman-Keuls test if overall p-value was <0.05, which was considered significant.

### EXAMPLE 2

The following example describes the properties of non-limiting compositions comprising a plurality of micelles and an anesthetic internally contained within the plurality of micelles.

Non-limiting micellar compositions were prepared by film dispersion, and liposomal compositions were prepared by thin film hydration. TEM images showed that DSPE-PEG (2000) micelles without drug loading were about 10 nm in diameter. After loading with bupivacaine, the diameter of the micelles increased to around 15 nm, as shown in FIG. 2B). Liposomes were ˜100 nm in diameter without and with loading with bupivacaine, also shown in FIG. 2B. Estimations of size obtained by DLS were similar, as shown in Table 1. The loading of bupivacaine in the micellar compositions was 10.9+/−0.5% (w/w), with an encapsulation efficiency of 65.6+/−2.7%. For the liposomal compositions, the bupivacaine loading and encapsulation efficiency were 14.2+/−0.9% and 16.5+/−1.1%, respectively, as shown in Table 1. The micellar composition comprising bupivacaine and liposomal composition comprising bupivacaine had a slight negative charge (˜−3 mV) on surface, also shown in Table 1.

TABLE 1
Composition properties.

| | | | Zeta | Drug | Encapsulation |
|---|---|---|---|---|---|
| | Diameter | Polydispersity | Potential | Loading | Efficiency |
| Composition | (nm) | index | (mV) | (%) | (%) |
| Micellar | 10.2 +/− 0.3 | 0.1 | −2.3 +/− 0.2 | — | — |
| Micellar | 15.1 +/− 0.4 | 0.1 | −3.2 +/− 0.2 | 10.9 +/− 0.5 | 65.6 +/− 2.7 |
| comprising | | | | | |
| bupivacaine | | | | | |
| Liposomal | 100.2 +/− 4.5 | 0.2 | −3.4 +/− 0.2 | — | — |
| Liposomal | 102.2 +/− 5.1 | 0.2 | −3.3 +/− 0.3 | 14.2 +/− 0.9 | 16.5 +/− 1.1 |
| comprising | | | | | |
| bupivacaine | | | | | |

### EXAMPLE 3

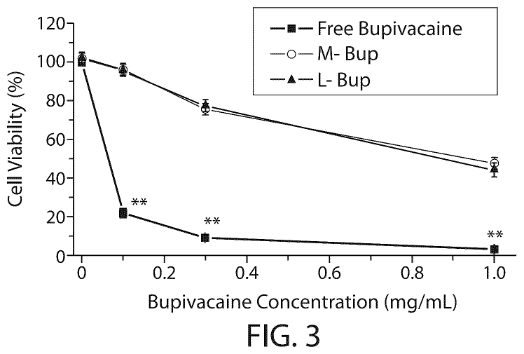

The following example describes the cytotoxicity of a non-limiting composition comprising a plurality of micelles and an anesthetic internally contained within the plurality of micelles. The cytotoxicity of the various compositions (10 mg/mL) was evaluated in HUVECs, as shown in FIG. 3. Cell viability in untreated cells was defined as 100%. In the absence of bupivacaine, compositions caused no cytotoxicity after 24 hours of incubation (101.8+/−3.2% and 102.5+/−2.2% cell survival for micelles and liposomes, respectively). Free bupivacaine was cytotoxic to the HUVECs: 22.1+/−2.4% cells were alive after incubation with 0.1 mg/mL free bupivacaine for 24 hours. In cells treated with micellar compositions comprising bupivacaine and liposomal compositions comprising bupivacaine, toxicity was markedly reduced compared to cells treated with the same concentration of free bupivacaine. For example, even at micellar compositions comprising 1 mg/mL bupivacaine, cell survival was ˜50%.

### EXAMPLE 4

The following example describes the interaction of a non-limiting composition comprising a plurality of micelles and an anesthetic internally contained within the plurality of micelles with cells.

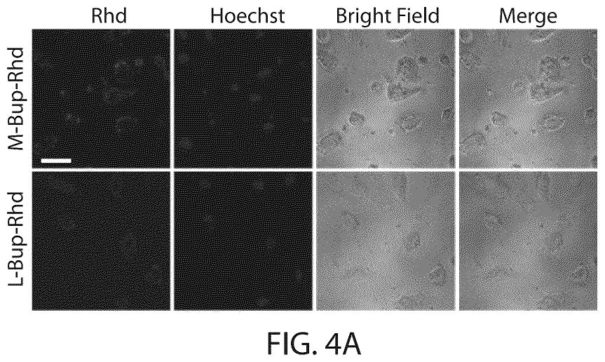

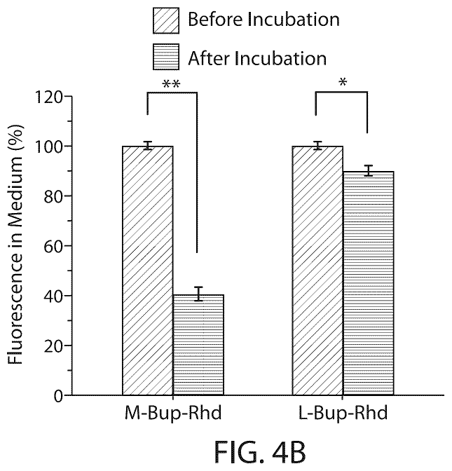

To investigate the interaction of non-limiting micellar compositions comprising bupivacaine with cells, Rhd-labeled compositions were incubated with HUVECs for 15 minutes at 4° C., which prevents endocytosis. The duration of exposure was selected to resemble the duration of tourniquet application in the rat Bier block model. After exposure to particles, cells were fixed with 4% PFA and imaged by CLSM. Strong fluorescence was seen with cells exposed to Rhd-labeled micellar compositions comprising bupivacaine (FIG. 4A, scale bar=50 micrometers), while little (˜10% of the fluorescence in the Rhd-labeled micellar compositions comprising bupivacaine, calculated from images using ImageJ) was detected in the group exposed to liposomal compositions comprising bupivacaine (FIG. 4A). The fluorescent intensity in the medium (e.g., not on or in cells) containing micellar compositions comprising bupivacaine decreased by 60% during the incubation with cells. Fluorescence in medium containing liposomal compositions comprising bupivacaine decreased by 10%, suggesting less association with cells, as shown in FIG. 4B, wherein data are means+/−S.D., n=4.

### EXAMPLE 5

The following example describes the effect of tourniquet time on anesthesia. Injury of a subject may be caused by too long a tourniquet time. A too short tourniquet time, however, might not result in adequate anesthesia, or in excessive toxicity. To find an acceptable tourniquet time, 0.4 mL of 0.5% bupivacaine was administered by IVRA after the tourniquet was applied, and the tourniquet was released in 5-10 minutes (Table 2, data are means+/−SD; n=4). A tourniquet time of 15 minutes resulted in anesthesia in four of four animals, with a mean duration of 2 hours. Prolonging tourniquet time to 20 minutes did not improve anesthesia, but shortening it affected anesthesia adversely. Consequently, a 15 minute tourniquet time was used in all subsequent experiments.

TABLE 2
Anesthesia duration resulting from different tourniquet times.

| | Successful | Anesthesia |
|---|---|---|
| Time (min) | Anesthesia (%) | Duration (h) |
| 5 | 0 | 0 |
| 10 | 25 | 0.3 +/− 0.5 |
| 15 | 100 | 2.0 +/− 0.6 |
| 20 | 100 | 1.9 +/− 0.5 |
| 30 | 100 | 2.0 +/− 0.5 |

### EXAMPLE 6

The following example describes the retention of a non-limiting composition comprising a plurality of micelles and an anesthetic internally contained within the plurality of micelles.

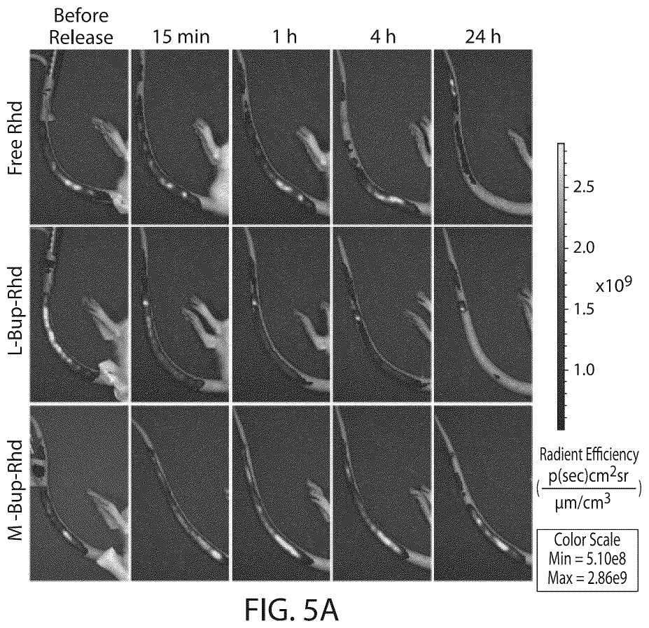

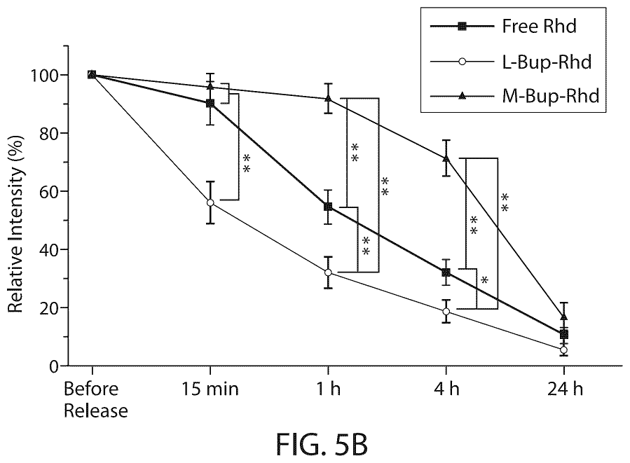

Free rhodamine, micellar compositions comprising bupivacaine, and liposomal compositions comprising bupivacaine were administered to rats intravenously according to the Bier block procedure, with the tourniquet on. Before releasing the tourniquet, rats were imaged by an In-Vivo Imaging System (IVIS), and the fluorescence intensity for each animal was considered 100% for subsequent calculations (fluorescence readings at subsequent time points were normalized to that value). After 15 minutes, the tourniquet was released and images were taken at 15 minutes, 1 hour, 4 hours, and 24 hours (FIG. 5A). FIG. 5B shows the quantification of the percentage of fluorescence intensity in the rat tail, wherein data are means+/−SD; n=4. In the groups treated with free Rhd, the fluorescence intensity gradually decreased, with ˜50% remaining after one hour. In the groups treated with Rhd-labeled liposomal compositions comprising bupivacaine, fluorescence in dropped more rapidly, to 56.1+/−7.2% in 15 minutes, and only ˜30% was left after one hour. In contrast, in the groups treated with Rhd-labeled micellar compositions comprising bupivacaine, fluorescence did not decrease significantly in the first hour after tourniquet release, remaining at 91.8+/−5.2%. Even after 4 hours, over 70% of intensity was maintained in the tail.

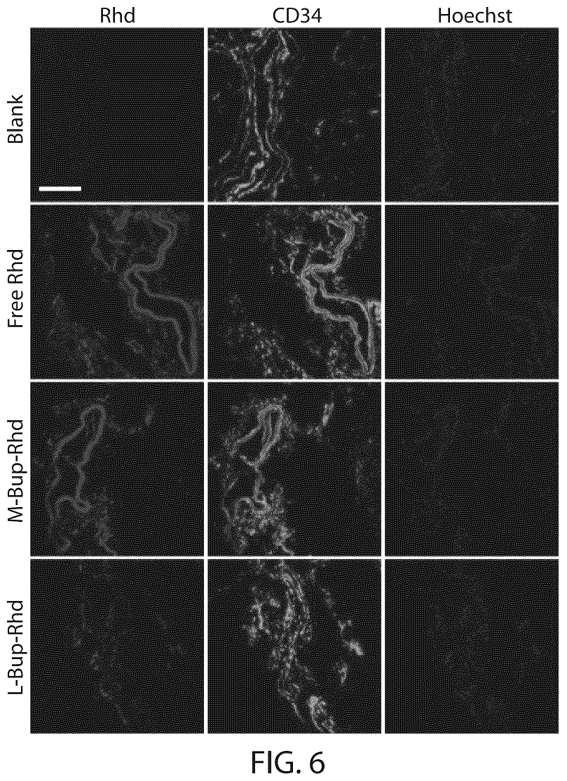

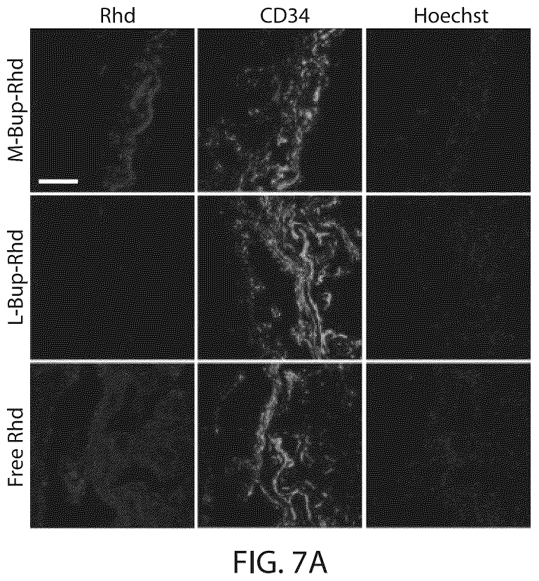

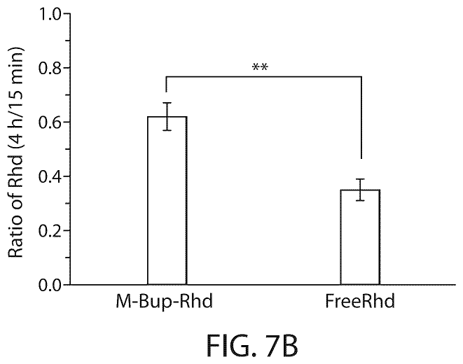

In a separate experiment, animals were euthanized 15 minutes after release of the tourniquet, tissues frozen, and sections analyzed by fluorescence microscopy, as shown in FIG. 6 wherein the scale bar is 200 micrometers. In groups treated with free Rhd and Rhd-labeled micellar compositions comprising bupivacaine, very strong Rhd signals co-localized with blood vessel (FIG. 6), identified by staining with anti-CD34 antibody. Little signal was seen in the groups treated with Rhd-labeled liposomal compositions comprising bupivacaine. Four hours later, Rhd-labeled micellar compositions comprising bupivacaine was still localized in blood vessels with strong Rhd intensity, while liposomal compositions comprising bupivacaine were almost undetectable, as shown in FIG. 7A, wherein the scale bar is 200 micrometers. In the free Rhd treated group, fluorescence was much weaker than at 15 minutes, calculated from images using ImageJ. FIG. 7B shows the ratio of total Rbd signal for a non-limiting micellar composition comprising bupivacaine at 4 hours (as shown in FIG. 7A) to 15 minutes (as shown in FIG. 6).

### EXAMPLE 7

The following example describes the duration of intravenous regional anesthesia after administering a non-limiting composition comprising a plurality of micelles and an anesthetic internally contained within the plurality of micelles.

The IVRA procedure was performed with 0.4 mL of compositions containing bupivacaine or saline, followed by neurobehavioral testing with a tail-flick analgesia meter, wherein the time (latency) the rat takes to respond to a stimulus with a tail flick is measured, and a longer latency indicates deeper anesthesia. The maximum latency is 18 seconds; baseline in saline-treated animals was ˜6 seconds (as it was in untreated animals). Free bupivacaine (0.5% w/v; 5 mg/mL in saline) was used. That concentration of bupivacaine could not be achieved with micellar compositions comprising bupivacaine or liposomal compositions comprising bupivacaine without the solutions becoming too viscous to inject, so they were prepared with 0.1% bupivacaine (w/v, 1 mg/mL). Consequently, a second group containing 0.1% free bupivacaine was also tested.

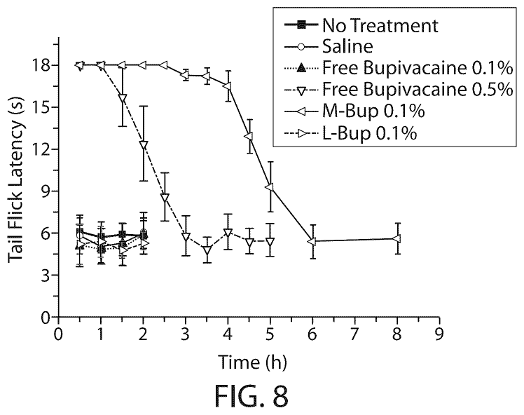

Tail analgesia from the group treated with 0.5% free bupivacaine lasted 2.0+/−0.6 hours. Tail analgesia in the group treated with a micellar composition comprising 0.1% bupivacaine group lasted more than twice longer, 4.5+/−0.5 hour, even though the bupivacaine dose was one fifth. Administration of 0.1% free bupivacaine or the liposomal composition comprising bupivacaine did not achieve any anesthetic effect (e.g., comparable to saline), as shown in FIG. 8 (data are means+/−SD; n=4) and Table 3.

TABLE 3
Frequency of successful anesthesia and duration
of anesthesia from each composition.

| | Bupivacaine | Successful | Anesthesia |
|---|---|---|---|
| Composition | Concentration (%, w/v) | Anesthesia (%) | Duration (h) |
| Free | 0.5 | 100 | 2.0 +/− 0.6 |
| bupivacaine | 0.1 | 0 | 0 |
| M-Bup | 0.1 | 100 | 4.5 +/− 0.5 |
| L-Bup | 0.1 | 0 | 0 |

### EXAMPLE 8

The following example describes the pharmacokinetics of bupivacaine after administering a non-limiting composition comprising a plurality of micelles and an anesthetic internally contained within the plurality of micelles.

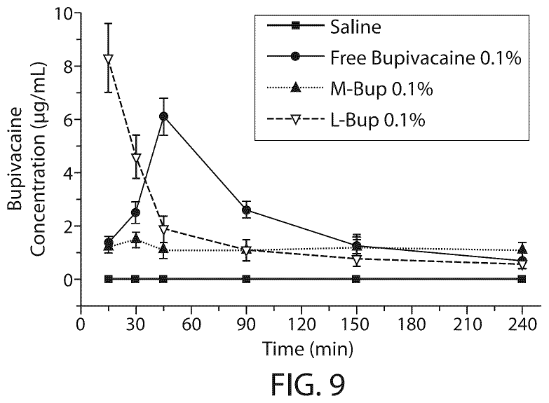

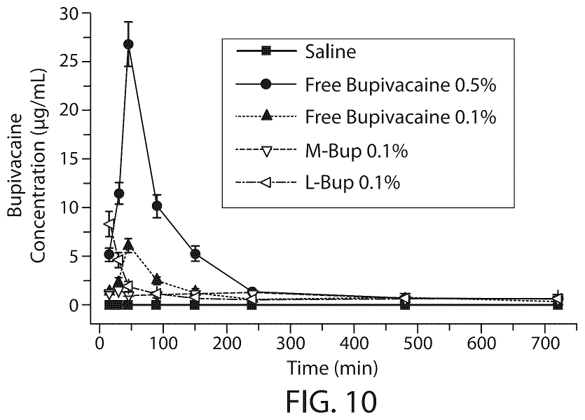

For each composition, the bupivacaine concentration in blood was studied after releasing the tourniquet (FIG. 9). In the group treated with 0.1% free bupivacaine, the drug concentration increased initially to a peak of 6.1+/−0.7 micrograms/mL at 45 minutes, then declined. In the group treated with 0.5% free bupivacaine, the peak bupivacaine concentration reached (26.8+/−2.3 micrograms/mL) (FIG. 10), and in this group, rats had a dyspnea although none died. In the group treated with liposomal compositions comprising 0.1% bupivacaine, the bupivacaine concentration was 8.3+/−1.3 micrograms/mL at the first time point (15 minutes), and then dropped rapidly. In group treated with micellar compositions comprising 0.1% bupivacaine, the bupivacaine concentration remained low, at ˜1.2 micrograms/mL).

### EXAMPLE 9

The following example describes the evaluation of tissue toxicity after administering a non-limiting composition comprising a plurality of micelles and an anesthetic internally contained within the plurality of micelles.

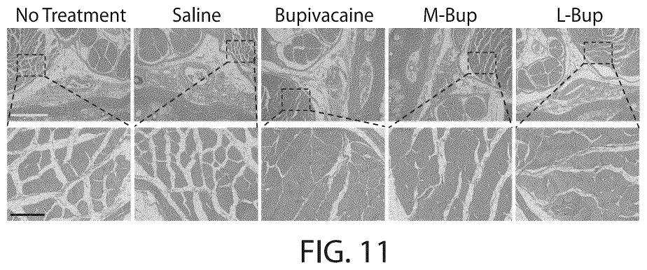

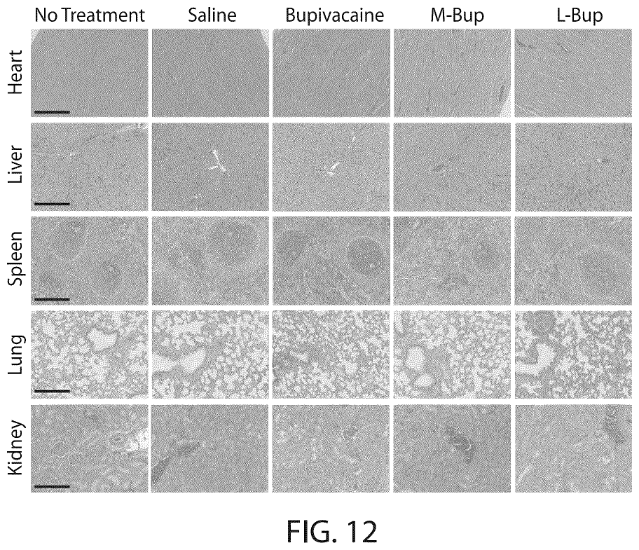

Twenty-four hours after the IVRA procedures, animals treated with saline, 0.5% free bupivacaine, liposomal compositions comprising 0.1% bupivacaine, and micellar compositions comprising 0.1% bupivacaine and were euthanized, and the tails and organs (heart, liver, spleen, lung and kidney) were harvested and processed into H&E-stained sections. Tissues from untreated rats were used as negative controls. Tail myotoxicity was evaluated as it is an easily identified manifestation of local anesthetic tissue toxicity. There was no myotoxicity in any group (FIG. 11, wherein the top scale bar is 200 micrometers and the bottom scale bar is 50 micrometers). Similarly, there were no histological abnormalities in the organs examined (FIG. 12, wherein all scale bars are 200 micrometers).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used berein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used berein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including otber elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-29. (canceled)

30. A method of delivering an anesthetic to a subject, comprising:
decreasing blood circulation in an extremity of a subject by at least 50%;
intravenously administering a composition comprising a plurality of micelles and/or particles to the extremity, the micelles and/or particles internally containing an anesthetic, wherein at least 50 mass % of the micelles and/or particles attach to a blood vessel surface within the extremity; and
restoring blood circulation in the extremity of the subject.

31. (canceled)

32. The method of claim 30, wherein the PEGylated lipids and/or polymers comprise DSPE-PEG, DPPE-PEG, DMPE-PEG, PEG-PLGA, and/or PEG-PLA.

33. The method of claim 30, wherein the PEGylated lipids and/or polymers have a molecular weight in the range of 200 Daltons to 5,000 Daltons.

34. The method of claim 30, wherein the PEGylated lipids and/or polymers comprise DSPE-PEG(2000).

35. The method of claim 30, wherein the micelles and/or particles have an average cross-sectional diameter of less than or equal to 30 nm.

36. The method of claim 30, wherein the micelles and/or particles have an average cross-sectional diameter of less than or equal to 20 nm.

37-39. (canceled)

40. The method of claim 30, wherein the blood vessel surface is a blood vein surface.

41-43. (canceled)

44. The method of claim 30, wherein the method is intravenous regional anesthesia.

45. The method of claim 44, wherein the intravenous regional anesthesia lasts for at least 4 hours.

46. The method of claim 30, wherein the micelles and/or particles release 90% of the local anesthetic into the extremity.

47. The method of claim 30, wherein decreasing blood circulation in the extremity of the subject comprises applying a tourniquet to the extremity of the subject.

48. The method of claim 47, wherein restoring blood circulation in the extremity of the subject comprises removing the tourniquet from the extremity of the subject.

49. (canceled)

50. The method of claim 48, wherein upon removing the tourniquet from the extremity, the concentration of anesthetic in blood of the extremity is less than or equal to 2 mg/mL.

51. The method of claim 30, wherein the anesthetic comprises bupivacaine.

52. The method of claim 30, wherein the anesthetic comprises lidocaine.

53. The method of claim 30, wherein the composition has a surface area-to-volume ratio of greater than about 0.10 and less than about 0.70.

54. (canceled)

55. The method of claim 30, wherein the at least 50 mass % of the micelles and/or particles attach to the blood vessel surface due at least in part to electrostatic interactions and/or hydrogen-bonding between the micelles and/or particles and the blood vessel surface.

56. The method of claim 30, wherein the at least 50 mass % of the micelles and/or particles attach to the blood vessel surface due at least in part to the average cross-sectional diameter of less than or equal to 30 nm.

57. The method of claim 30, wherein the micelles and/or particles release the anesthetic upon attaching to the blood vessel surface.

58. A method of delivering an anesthetic to a subject, comprising:
applying a tourniquet to an extremity of the subject;
administering a composition to the extremity, the composition comprising a plurality of micelles and/or particles having an average cross-sectional diameter of less than or equal to 30 nm, and internally containing an anesthetic; and
removing the tourniquet from the extremity.

59-64. (canceled)
